(19) United States
(12) Patent Application Publication
(10) Pub. No.: US 2018/0125084 A1
(43) Pub. Date: May 10, 2018

(54) Forced Air Ozone Reactor for Microbial Reduction

(71) Applicant: HARPC Solutions Inc., Beamsville (CA)

(72) Inventors: Paul Moyer, Beamsville (CA); Mark VanderVeen, Burlington (CA)

(21) Appl. No.: 15/642,996

(22) Filed: Jul. 6, 2017

Related U.S. Application Data
(60) Provisional application No. 62/406,199, filed on Oct. 10, 2016.
(60) Provisional application No. 62/362,779, filed on Jul. 15, 2016.

(51) Int. Cl. A23B 7/152; A23L 3/3409; A23L 3/3445

(52) U.S. Cl. CPC A23B 7/152; A23V 2002/00; A23L 3/3445; A23L 3/34095

(57) ABSTRACT

Disclosed is an apparatus for inactivating bacteria and/or reducing microbial count on a food product or a container therefore, which is susceptible to surface and sub-surface microbial presence is provided, said apparatus comprising a sealable chamber which is operably connected to i) an ozone generator for generating ozone gas, and ii) an evacuation fan for forcing movement of ozone gas through the sealable chamber. Also disclosed is a method for inactivating bacteria and/or reducing microbial count on a food product or a container therefore, which is susceptible to surface and sub-surface microbial presence is provided, said method comprising a) providing a plurality of said food product or container in a sealable chamber which is operably connected to i) an ozone generator for generating ozone gas, and ii) an evacuation fan for forcing movement of ozone gas vertically through the sealable chamber; b) creating condensation on surface of the food product or container by adjusting humidity in the sealable chamber to reach a predetermined humidity; c) operating the ozone generator and the evacuation fan to generate a predetermined exhaust air velocity to pass ozone gas generated by the ozone generator through the sealable chamber for a predetermined period of dwell time; and d) expelling ozone gas from the sealable chamber. The present invention further provides for a method for reducing a level of bacteria, yeast, mold and mildew in or on a container.

### FIELD OF THE INVENTION

The present invention relates generally to methods and apparatuses for reducing microbial count in food and containers therefor. The methods and apparatuses of the present invention are described herein with reference to apples in order to facilitate understanding of the invention. However, it should be clear to those skilled in the art that applicability of said methods and apparatuses is not limited to apples. Rather, said methods and apparatuses can be adapted to reduce microbial count in other products susceptible to undesirable surface and sub-surface microbial presence, such as other fruits and vegetables, beehives, as well as containers therefor.

### DISCUSSION AND COMPARISON WITH RELEVANT PRIOR ART

In December 2014, a multistate listeriosis outbreak in the United States was linked to consumption of caramel-coated apples. Over the next few months, an investigation revealed that the Listeria originated on the surface of the affected apples, which were subsequently introduced into the interior of the apples when sticks to be used as handles punctured the apples during production. Although risk of listeriosis from candy apples can still be regarded as low, there is a need to apply preventative measures during caramel apple production.

Washing apples in aqueous sanitizers is one example of such preventative measure. However, water wash systems are not always practical due to cost and space limitations as well as concerns about bringing water into a manufacturing facility. Further, this sanitizing option was found to have limited efficacy in removing contamination (<1 log cfu reduction) and potentially can lead to cross-contamination (Perez-Rodriguez et al., 2014, “Study of the cross-contamination and survival of Salmonella in fresh apples”, International Journal of Food Microbiology, 184, 92-97, the entire disclosure of which is incorporated herein by reference). In addition, residual moisture on apples impedes coating of caramel on apples thereby creating difficulties during production. Consequently, aqueous free approaches (for example, hydrogen peroxide vapor) are more compatible with candy apple production and moreover, have proven to be effective in decontaminating produce when compared to traditional post-harvest washing (Back et al., 2014, “Effect of hydrogen peroxide vapor treatment for inactivating Salmonella Typhimurium, Escherichia coli 0157:H7 and Listeria monocytogenes on organic fresh lettuce.” Food Control, 44, 78-85, the entire disclosure of which is incorporated herein by reference).

Ozone has been associated with antimicrobial activities and designated as Generally Recognized as Safe (GRAS) by the U.S. Food and Drug Administration. (See, e.g., Sharma and Hudson, “Ozone gas is an effective and practical antibacterial agent”, Am J Infect Control. 2008 Oct; 36(8): 559-63, the entire disclosure of which is incorporated herein by reference). Processes of using solution containing ozone for decontaminating food are described in, e.g., U.S. Pat. Nos. 6,485,769 and 6,162,477. However, water is often the source of contamination in food manufacturing facilities. Moreover, as noted above aqueous free approaches are more compatible with certain types of food products including candy apples.

More recently, use of ozone gas was suggested. (See, e.g., Khadre et al., 2001, “Microbiological aspects of ozone applications in food: A review”, Journal of Food Science, 66, 1262-1252, the entire disclosure of which is incorporated herein by reference). Previous studies have demonstrated that ozone introduced into the atmosphere of storage rooms can reduce microbial loading on fruit (Yaseen et al., 2015, “Ozone for post-harvest treatment of apple fruits”, Phytopathologia Mediterranea, 54, 94-103, the entire disclosure of which is incorporated herein by reference). However, ozone in storage rooms is applied at a low level (0.5-2 ppm) to prevent excessive corrosion of fittings and reduce hazards to workers. Consequently, an extended exposure time is required to achieve microbial reductions although contacting each individual apple represents a challenge.

The present invention relates to methods and apparatuses which use gaseous ozone introduced by forced air flow to reduce microbial, in particular Listeria count, in food such as fruits and vegetables, beehives as well as containers therefor. The present invention can also be adapted to reduce total aerobic count and yeast and mold levels on the surface of plastic containers. According to the present invention, ozone is introduced using forced air, making it possible to use higher ozone concentrations and facilitating controlled, even (as opposed passive) air flow through a container of a plurality of objects, such as apples. An added advantage is obtained when introducing the ozone at early stages of the drying portion of the apple processing system. The relative humidity surrounding the fruit is high at this stage, so that in theory the susceptibility of microbial cells to the lethal effects of ozone is increased (Miller et al., 2013, “A review on ozone-based treatments for fruit and vegetables preservation”, Food Engineering Reviews, 5, 77-106 and de Candia et al., 2015, “Eradication of high viable loads of Listeria monocytogenes contaminating food-contact surfaces. Frontiers in Microbiology, 6, 12 the entire disclosure each is incorporated herein by reference).

### SUMMARY OF THE INVENTION

The present invention provides for a method for inactivating bacteria and/or reducing microbial count on a food product susceptible to surface and sub-surface microbial presence, or a container therefor, said method comprising providing a plurality of said food product or container in a sealable chamber which is operably connected to i) an ozone generator for generating ozone gas, and ii) an evacuation fan for forcing movement of ozone gas vertically through the sealable chamber; creating condensation on surface of the food product or container by adjusting humidity in the sealable chamber to reach a predetermined humidity; operating the ozone generator and the evacuation fan to generate a predetermined exhaust air velocity to pass ozone gas generated by the ozone generator through the sealable chamber for a predetermined period of dwell time sufficient to kill 99-99.999% of the bacteria; and expelling ozone gas from the sealable chamber.

The present invention further provides for a method for reducing a level of bacteria, yeast, mold and mildew in or on a container, said method comprising providing one or more of said containers in a sealable chamber which is operably connected to i) an ozone generator for generating ozone gas, and ii) an evacuation fan for forcing movement of ozone gas vertically through the sealable chamber; creating condensation on surface of the container or containers by adjusting humidity in the sealable chamber to reach a predetermined humidity; operating the ozone generator and the evacuation fan to generate a predetermined exhaust air velocity to pass ozone gas generated by the ozone generator through the sealable chamber for a predetermined period of dwell time; and expelling ozone gas from the sealable chamber.

### BRIEF DESCRIPTION OF THE DRAWINGS

FIG. 1: shows an illustration of an apparatus in accordance with an embodiment of the invention as described herein.

FIG. 2: illustrates the step 1 (condensation) of a method for reducing microbial count in apples in accordance with an embodiment of the present invention. Ozone slide gate closed. Bins of apples wrapped and hood down. Door open. Run dryer exhaust fan to draw warm air up through apples and create surface condensation.

FIG. 3: illustrates the step 2 (ozonation) of a method for reducing microbial count in apples in accordance with an embodiment of the present invention. Ozone slide gate open. Bins of apples wrapped and hood down. Door closed. Ozone generator runs and ozone gas starts to sink downwards. Evacuation fan runs on low speed to disperse ozone gas through apples.

FIG. 4: illustrates the step 3 (evacuation) of a method for reducing microbial count in apples in accordance with an embodiment of the present invention. Ozone slide gate remains open. Bins of apples wrapped and hood down. Door closed. Ozone generator turns off. Evacuation fan runs on high speed to expel ozone gas from chamber, drawing fresh air in through dryer exhaust open duct and through ozone generator open duct.

FIG. 5: illustrates the step 4 (air drying) of a method for reducing microbial count in apples in accordance with an embodiment of the present invention.

FIG. 6: shows an illustration of a forced air ozone reactor of an apparatus in accordance with an embodiment of the invention as described herein.

FIG. 7: shows a schematic diagram of ozone treatment chamber and position of inoculated apples in accordance with experimental setup of EXPERIMENT 1 described herein.

FIG. 8: shows log reduction of Listeria monocytogenes and Lactobacillus inoculated onto apples then treated with ozone introduced at a rate of 6 g/h for different time periods. At five minutes of exposure ozone concentration measured 30 ppm±2, at ten minutes 55 ppm±2, and at fifteen minutes 77 ppm±2.

FIG. 9: shows the effect of exhaust fan velocity on the measured ozone concentration within the Treatment chamber.

FIG. 10: shows log reduction of Lactobacillus inoculated onto apples treated within the ozone chamber operating at different fan exhaust velocities. The inoculated apples were placed at different locations within the apple pile then treated for 20 mins.

FIG. 11: shows log reduction of Lactobacillus inoculated onto apples then treated with ozone within a reactor operating at 250 cfm or 500 cfm fan exhaust velocity. Each point represents an average of 5 apples located at different points within the apple pile.

FIG. 12: shows measured ozone concentration within the reactor operating at 500 cfm. Five different runs having varying stopping points are shown.

FIG. 13: shows schematic of dryer system used in EXPERIMENT 1. Apple drying procedure: wrap side of bins to be dried in stretchwrap. Place under hood, ensuring a good seal. Turn on fan and dry apples.

FIGS. 14A and 14B: show graphs of temperature profiles of sub-surface of apples at the top or bottom of the apple column in the laboratory scale reactor. Ozone was introduced at the top and drawn through the apple pile. The ambient temperature within the reactor was 23° C.

FIG. 15: shows a graph of the total aerobic count and yeast and mold count of non-inoculated Reusable Plastic Containers (RPC's) that were treated within the forced air ozone reactor compared to non-treated controls in EXPERIMENT 2.

FIG. 16: shows a graph of the effect of treatment time on the log reduction of Lactobacillus inoculated onto apples then treated within a forced air ozone reactor. The apples were positioned at the top, middle and bottom of the apple pile (2 bins) then treated with ozone using an air exhaust fan speed of 500 cfm, in EXPERIMENT 1.

### DESCRIPTION OF THE PREFERRED EMBODIMENT

While aspects of the invention described herein are described with reference to inactivating bacteria and/or reducing microbial count in fruit, in particular apples, it should be appreciated that the described methods, apparatuses and related assemblies can be used to reduce microbial count in other types of foods or products.

Further, specific embodiments and examples of the methods and apparatuses described herein are illustrative, and many variations can be introduced on these embodiments and examples without departing from the spirit of the disclosure or from the scope of the appended claims. Elements and/or features of different illustrative embodiments and/or examples may be combined with each other and/or substituted for each other within the scope of this disclosure and appended claims.

### Definitions

As used herein, and unless stated otherwise, each of the following terms shall have the definition set forth below.

As used herein, “about” in the context of a numerical value or range means ±10% of the numerical value or range recited or claimed. By any range disclosed herein, it is meant that all hundredth, tenth and integer unit amounts within the range are specifically disclosed as part of the invention. Accordingly, “about” a recited value specifically includes that recited value. For example, a range of about 20 minutes refers to all measurements within the range of ±10% of 20 minutes, including 20 minutes.

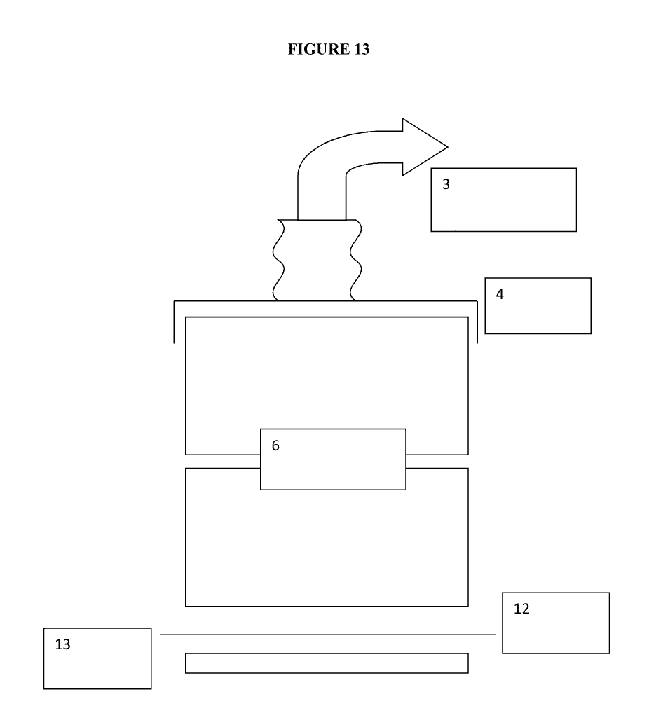

Through a series of experiments, the inventors of the methods and apparatuses described herein showed that Listeria can be killed on produce, in particular apples, by fumigating them with ozone gas. In this series of experiments, the results ranged from a 2-log to a 5-log kill. Each “log” reduction indicates the extent of the kill by a factor of 10. That is to say there was 99% (2-log) to 99.999% (5-log) kill of Listeria. These initial positive laboratory results suggested that a larger, commercial scale application test was warranted. Accordingly, an ozone chamber large enough to house 1600-3000 lbs of apples was built, which incorporated an apple dryer system developed by the inventors, and connected to an ozone generator, electrical controls and safety system. A schematic of said dryer system is shown in FIG. 13. Results from experiments conducted in said large-scale ozone chamber is discussed in EXPERIMENT 1.

Accordingly, in one embodiment of the present invention, an apparatus for inactivating bacteria and/or reducing microbial count on a food product susceptible to surface and sub-surface microbial presence, or a container therefor, is provided, said apparatus comprising a sealable chamber which is operably connected to i) an ozone generator for generating ozone gas, and ii) an evacuation fan for forcing movement of ozone gas vertically through the sealable chamber.

In an embodiment of the apparatus as described herein, the apparatus further comprises an ozone sensor. In another embodiment, the apparatus further comprises a dryer assembly, said dryer assembly comprising a hood and a dryer exhaust fan.

In an embodiment, the sealable chamber has capacity to hold 1-3000 lbs, preferably 10-3000 lbs of food product. In another embodiment, the sealable chamber has capacity to hold about 1600-3000 lbs of food product. In yet another embodiment, the sealable chamber has capacity to hold at least 1, at least 10, at least 100 or at least 200 lbs of food product.

In another embodiment of the present invention, a method for inactivating bacteria and/or reducing microbial count on a food product susceptible to surface and sub-surface microbial presence, or a container therefor is provided, said method comprising a) providing a plurality of said food product or container in a sealable chamber which is operably connected to i) an ozone generator for generating ozone gas, and ii) an evacuation fan for forcing movement of ozone gas vertically through the sealable chamber; b) creating condensation on surface of the food product or container by adjusting humidity in the sealable chamber to reach a predetermined humidity; c) operating the ozone generator and the evacuation fan to generate a predetermined exhaust air velocity to pass ozone gas generated by the ozone generator through the sealable chamber for a predetermined period of dwell time; and d) expelling ozone gas from the sealable chamber.

In an embodiment of the method as described herein, the bacteria is Listeria. In another embodiment, the bacteria is Salmonella or E. Coli.

In one embodiment, the food product is a fruit or a vegetable. In another embodiment, the food product is apple, melon, lettuce, e.g., shredded lettuce, mushroom, zucchini, cucumber or beehive. In yet another embodiment, the food product is seed, spice, tea, grain, dried fruits, or nuts. In a further embodiment, the food product includes processed foods.

In one embodiment, the predetermined humidity is about 70-100% or about 65-85%, preferably about 80-90% or about 85%. In another embodiment, the dwell time is greater than 10 minutes. In yet another embodiment, the dwell time is between about 20 minutes and about 40 minutes, specifically, about 20 minutes or about 40 minutes.

In one embodiment, the predetermined exhaust air velocity is about 10-1500 cfm. In another embodiment, the predetermined exhaust air velocity is about 250-700 cfm, preferably about 300-600 cfm or about 500 cfm.

In one embodiment, ozone concentration in the sealable chamber is maintained at about 4-20 ppm in step c) for a period of time sufficient to kill from 99-99.999% of the bacteria. In another embodiment, ozone concentration in the sealable chamber is maintained at about 14-20 ppm or 4-6 ppm in step c) for a period of time sufficient to kill from 99-99.999% of the bacteria.

In one embodiment the sealable chamber has capacity to hold 1-3000 lbs, preferably 10-3000 lbs of food product. In another embodiment, the sealable chamber has capacity to hold about 1600-3000 lbs of food product. In another embodiment, the sealable chamber has capacity to hold at least 1, at least 10, at least 100 or at least 200 lbs of food product.

In one embodiment of the method as described herein, said method excludes a step of contacting an ozone-containing liquid with the food product or container. In another embodiment, ozone is introduced into the sealable chamber at rates of about 1-60 g/h, preferably about 6-60 g/h.

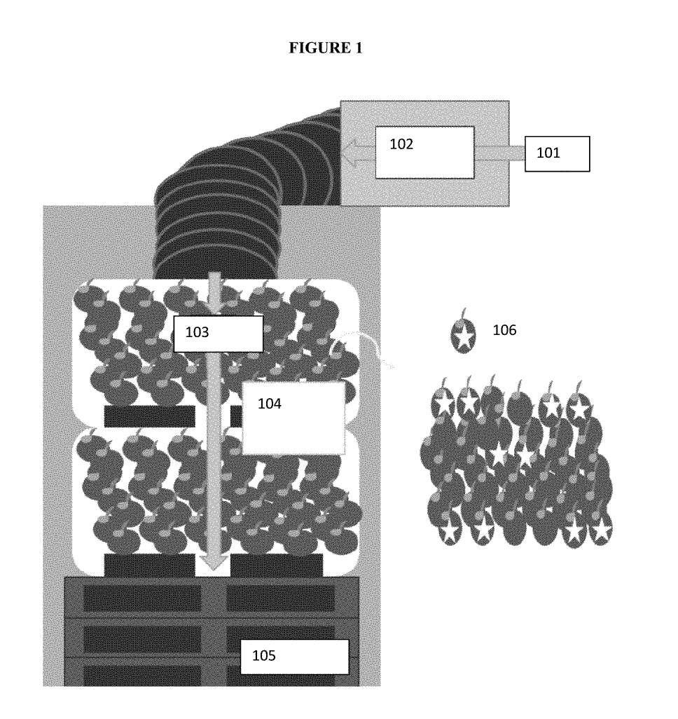

In an embodiment, an apparatus is provided comprising a sealable chamber, an ozone generator and a (preferably) two-speed evacuation fan. The sealable chamber can also comprise an interior ozone sensor connected to a room exhaust fan. The apparatus can further comprise a dryer equipment assembly comprising of hood seated on a top bin of the food to be sanitized and a dryer exhaust fan. An illustration of said apparatus is shown in FIG. 1. In FIG. 1, the following legends are used: 101: oxygen gas; 102: ozone generator; 103: ozone gas; 104: apple bins (open at bottom); 105: ventilation; 106: inoculated apples.

In another embodiment, the method and apparatuses as described herein can be adapted to reduce Pseudomonas in biofilms and on the surface of containers such as Reusable Plastic Containers (RPCs) to low levels detectable only by enrichment. In a further embodiment, the method and apparatuses as described herein can be adapted to reduce total aerobic count and yeast and mold counts in containers, in particular plastic containers and containers for food and beehives. It is also envisioned within the scope of the present invention to reduce microbial count in containers and food products contained therein at the same time (in a single run).

In another embodiment, the method and apparatuses as described herein can be adapted to reduce pesticides on food products.

The steps of a method for reducing microbial count in food products and containers using said apparatus, in accordance with an embodiment of the present invention, is described below.

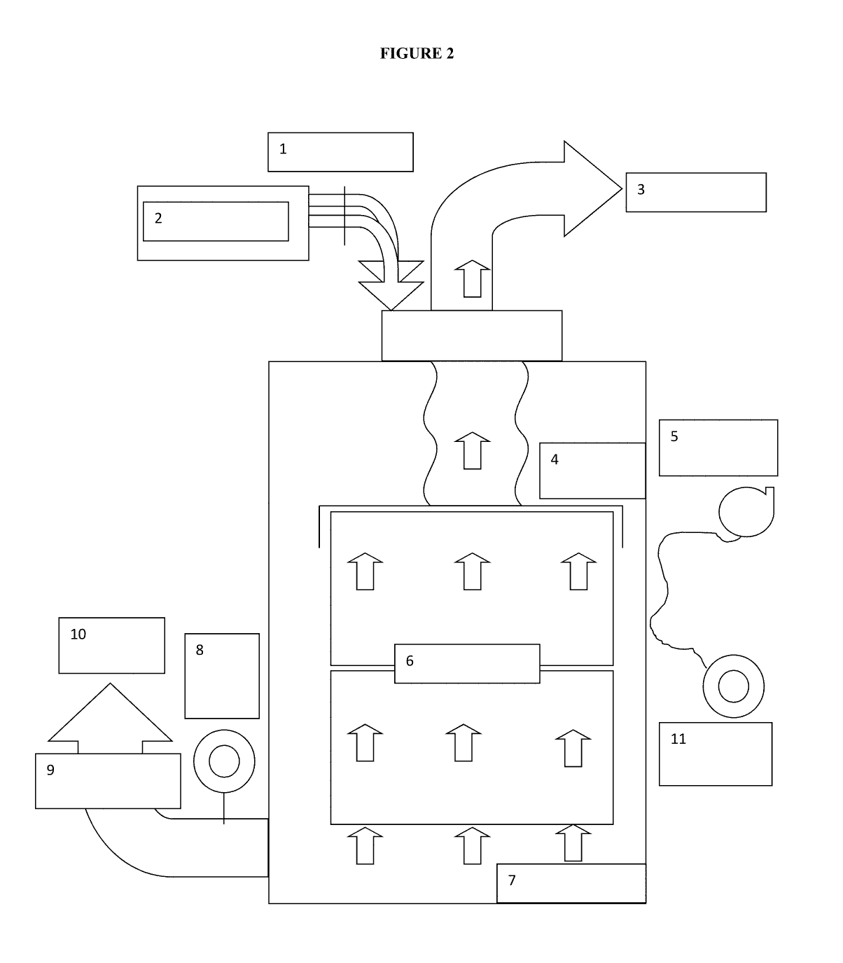

The following legends are used for FIGS. 2-5 and 13:
1: ozone slide gate
2: ozone generator
3: dryer exhaust fan
4: adjustable hood
5: room exhaust fan
6: bin of apples
7: sealed/sealable chamber
8: internal ozone sensor
9: evacuation fan
10: carbon filter
11: room ozone sensor
12: plastic sheet
13: plastic pallet
   Step 1: Condensation (illustrated in FIG. 2)

Bins of apples (6) are taken out of a cooler (temperature 36-40° F.) and are wrapped from top to bottom in clear plastic wrap to ensure they are air-tight. The top of the stack of bins is left open as is the underside of the bottom bin. It is noted that this particular arrangement may be varied to achieve a similar effect. In other words, the produce is retained within a preferably vertical container which is closed to the environment on its perimeter and open at the top and bottom. For experimental purposes, the container was formed herein by stacking open-ended bins and sealing them about the respective perimeters at their connecting points. The wrapped bin stack is placed in the sealable chamber (7) and the hood (4) sealingly lowered onto the top bin using a system of pneumatic cylinders. A tight fit is important so that the apples 2(6) cannot be bypassed. The ozone generator slide gate (1) is closed. The chamber door is left open. The dryer exhaust fan (3), which is in fluid connection with the bin stack via the hood (4), is run (in this case, at 2400 cfm) to draw warm air up through the open bottom of the bin stack and through the apples (6) to create surface condensation on the apples (6). The fan is run (in this case for 10-15 minutes) to reach the desired humidity of 70-100%, preferably about 80-90% or about 85% within the chamber. The dryer fan (3) is then turned off.

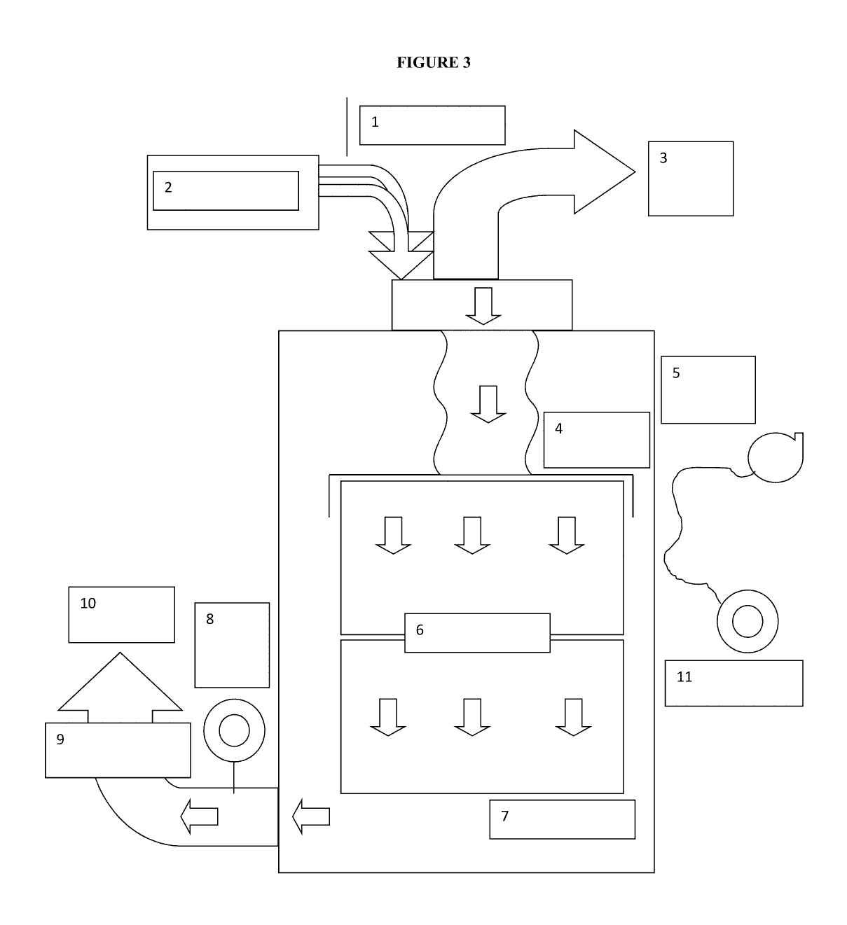

Step 2: Ozonation (illustrated in FIG. 3)

Once the desired humidity is reached and the dryer fan (3) is off, the chamber door is securely closed and latched. The ozone slide gate (1) is opened. The ozone generator (2) runs and ozone gas enters through the hood (4) and downward through the apple container. The ozone generator output (ozone rate) can be selected depending on the size of the ozononation chamber.

The evacuation fan (9) runs at the bottom of the chamber on low speed, e.g., 300-600 cfm, to disperse ozone gas through the apples and create negative pressure in the chamber. Air flow is directed through the bed of product to create pressure differential and turbulence. The speed of the evacuation fan should be selected depending on the product being ozonated.

Ozone is drawn down through the apples from top to bottom, then out through the evacuation fan (9) and the carbon filters (10) before being exhausted outside of the chamber. The dwell time for ozone will vary depending on the size of the apples, bin volume and how much ozone is sequestered by any organic compounds on the apples. An ozone sensor (8) inside the chamber near the evacuation fan (9) monitors the concentration of ozone gas once it has passed through the apples. During the ozonation process, exhaust fan speed is selected to achieve a concentration which varies between 14 and 20 ppm. Once the concentration stops climbing and the desired time of exposure has been achieved, the ozonation process is complete and the ozone generator (2) shuts off

Optionally, should ozone be detected in the room by the room sensor (11), there is an alarm and the room exhaust fan (5) will run

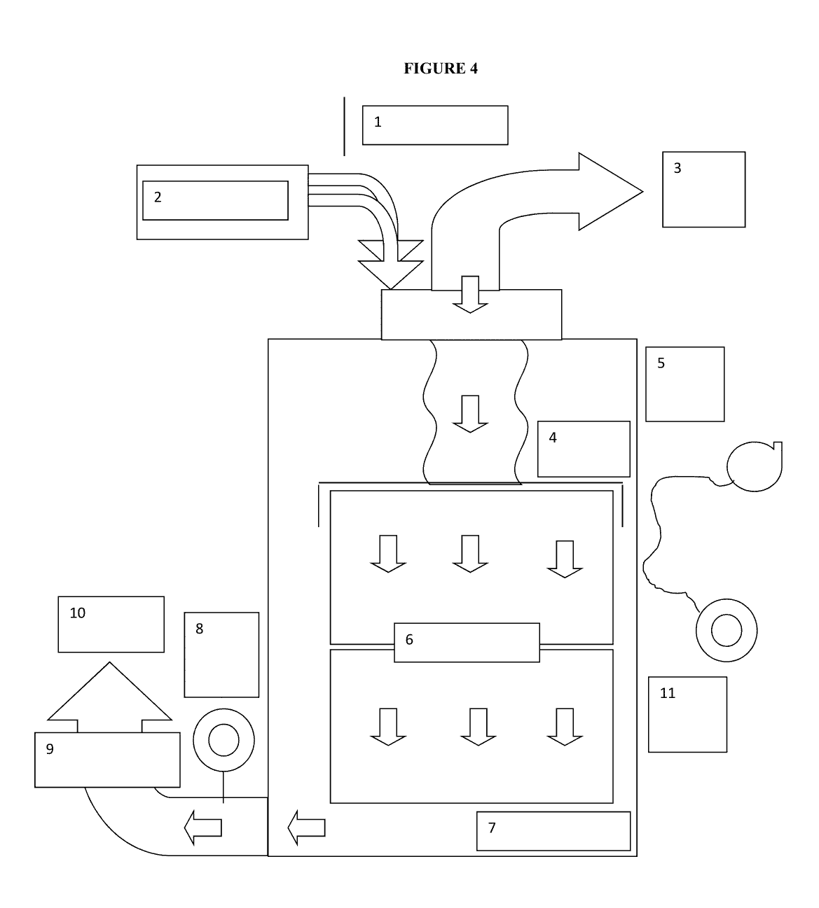

Step 3: Evacuation (illustrated in FIG. 4)

The ozone generator (2) turns off. The evacuation fan (9) runs on high speed at approximately 1000 cfm to expel ozone gas from chamber, drawing fresh air in through dryer exhaust open duct and through the ozone generator open duct. This takes approximately 40 seconds until the ozone sensor (8) inside the chamber reads 0.

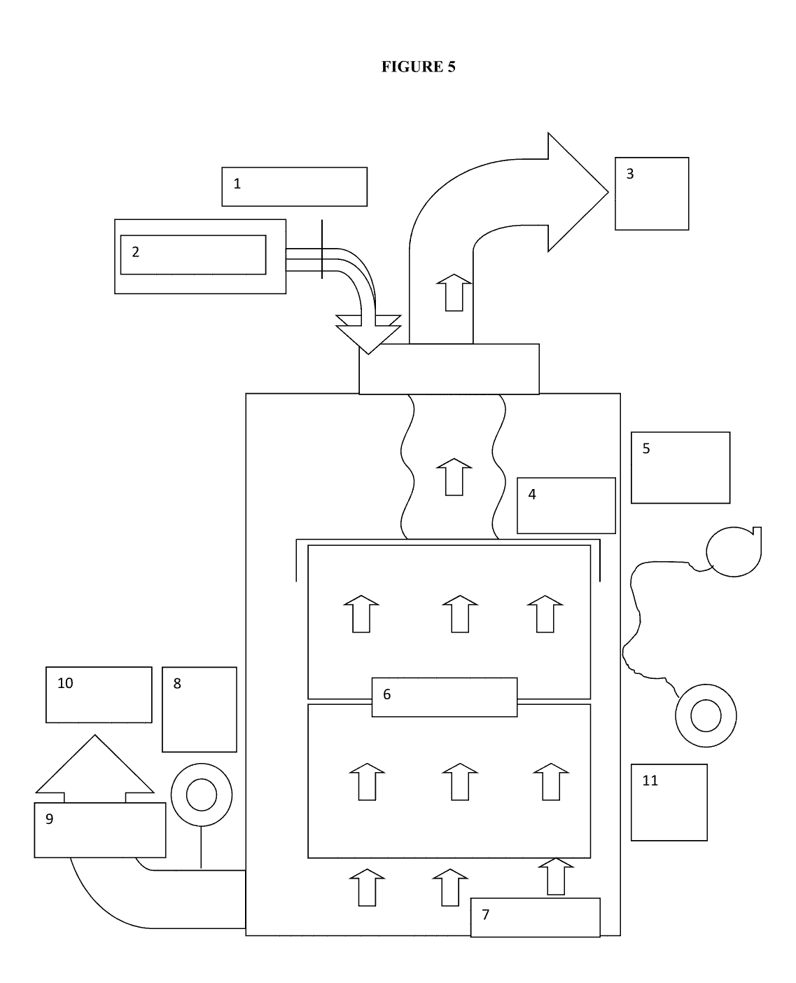

Step 4: Air Drying (illustrated in FIG. 5)

Once the ozone has been evacuated from the chamber, the door is opened and the ozone slide gate (1) closed. Bins of Apples (6) remain wrapped and hood (4) down. Run dryer exhaust fan (3) to draw warm air up through apples until at room temperature (90 minutes.)

Specific process parameters mentioned in the embodiment described above are provided as examples. A skilled person would recognize that many of the process parameters are interrelated. For example, the target ozone concentration during the ozonation step can vary depending on types of food product, batch size and size of ozone chamber. The airflow has to be sufficient to distribute the ozone generated by the ozone generator evenly through the product bed. Too low an air speed does not distribute the ozone evenly and achieves kill only at certain points in the product bed. The inventors have determined that 500 cfm airflow through 2400 lbs of 72 count size apples stacked in three 4′×4′×3′ bins will achieve homogenous distribution of ozone using a 60 g per hour (1 gram per minute) ozone delivery and optimum bacterial kill. This process can be scaled up and down.

In an embodiment, process parameters of the claimed method for inactivating bacteria on a food product susceptible to surface and sub-surface microbial presence is as follows:

### Ozone:

1) between 0.1 and 3 grams per minute in a 160 cubic foot container, corresponding to a ratio of 0.000625 g per cubic foot and 0.01875 g per cubic foot.
2) between 0.1 and 3 grams per minute into 2400 lbs of apples, corresponding to a ratio of 0.00004167 grams per minute per pound of food and 0.00125 grams per minute per pound of food.
3) between 0.1 grams and 3 grams per minute for 40 minutes, corresponding to a ratio of 4 grams to 120 g of ozone per 2400 lbs of apples.
4) Ozone concentration will vary depending on specific process parameters selected. For example, the inventors have found air flow at 500 cfm resulted in a peak ozone concentration of 4-6 ppm.
   Air flow: between 10 cfm and 1500 cfm through the bed of food product in a 160 cubic foot container, corresponding to a ratio of 0.0625 cfm per cubic foot of container and 9.375 cfm in air flow per cubic foot of container. Target air velocity can depend on the size of the sealable chamber.

Temperature: between 36° F. and 90° F.

Humidity: 70% and 100%.

Size of container/surface area of food: each 4′×4′×3′ bin holds 800 lbs apples or 1350 individual apples of 72 count size (72 apples in a bushel). Each apple weighs an average of 0.6 lbs (260 grams). The surface area of each apple is on average 37 in² (230 cm²). Therefore, in each bin 49,950 in² (310,500 cm²) of product surface is treated.

The above ratios can be used to calculate parameters for other types and sizes of food product, for example cherries, lettuce, and watermelons. Specifically, cherries have larger surface area than apples per unit weight. Watermelons have smaller surface area than apples per unit weight. Therefore, the ozone exposure required to achieve the desirable bacteria kill level would be higher for cherries, and lower for watermelons. The level of ozone exposure can be adjusted by, e.g., adjusting air flow and/or dwell time, and/or using sealable chambers of different sizes.

Use of sealable chambers having various sizes are within the scope of the methods and apparatuses described herein, including sealable chambers having a size corresponding to that of a standard microwave used with a small fan, and much larger units for bulk produce handlers.

Ozone dwell time: The dwell time depends on the volume of product and concentration of ozone. The inventors have found best results at 40 minute dwell time for three bins of apples at 800 lbs per bin.

An ozone monitor (5) can be optionally installed in the room, which is programmed to automatically shut off the chamber and start the room exhaust fan (5) if 0.1 ppm ozone is detected.

The apparatuses and methods of the present invention are advantageous over previously known sanitation methods in that it is eco-friendly. Specifically, the method of the subject invention does not use water, thereby conserving fresh water and avoids creation of chemical water effluent with harsh sanitizing chemicals like chlorine or ammonia. In addition, ozone gas decomposes into oxygen, leaving no dangerous or harmful by-products.

Finally, the combination of any embodiment or feature mentioned herein with one or more of any of the other separately mentioned embodiments or features is contemplated to be within the scope of the instant invention.

### EXPERIMENTS

### EXPERIMENT 1

### Development of Forced Air Ozone Reactor For Inactivating Listeria monocytogenes on Apples Destined For Candy Apple Production

### Summary

The efficacy of a forced air ozone reactor for decontaminating apples has been assessed using a combination of laboratory and commercial scale studies. In laboratory studies, the flow dynamics of ozone gas through apples inoculated with Listeria monocytogenes was studied. Under certain conditions it was possible to decrease Listeria levels on apples by 2.12-3.07 log cfu. A commercial scale unit with a capacity of treating two totes of apples (2000 lbs.) in a single run was constructed. To facilitate commercial trials a Lactobacillus strain was selected with equal resistance to ozone compared to Listeria. Through validation studies, it was found that the homogeneity and lethality of the treatment to inactivate the surrogate inoculated onto apples, was dependent on the air dynamics and treatment time. Under certain conditions it was possible to achieve a 4.42 log cfu reduction of the Listeria surrogate. In conclusion, it has been demonstrated that the forced air ozone treatment can be applied in a hurdle approach, to manage risk associated with Listeria in the course of apple processing.

### Materials and Methods

### Bacteria Used and Inoculation of Apples

The pathogens used in this study included Shiga toxin-producing E. coli—STEC, serotypes O157:H7 (two strains) and one strain of O111, O45, O26, as well as Listeria monocytogenes (serotypes 4a, 4b, 1/2b, 1/2a, and 3a). These isolates are of particular relevance as they are associated with past outbreaks, and were obtained from the University of Guelph's Food Science culture collection. Listeria monocytogenes serotype 4b (isolated from fresh produce) and Lactobacillus fructivorans (isolated from wine) in particular were used throughout the study. Lactobacillus fructivorans ATCC 8288 was also applied in the study as a surrogate for L. monocytogenes and obtained from American Type Culture Collection (Atlanta, US).

The concentration of each bacteria was determined by both optical density (OD) and serial dilution. After each bacterial was diluted to the same concentration (8-log ₁₀ CFU/ml) the E. coli strains were mixed together to make a final inoculum, as well as the L. monoctyogenes strains.

The inoculums were stored at 4° C. for up to 12 hours before use and vortexed for 1 minute once removed. Each bacteria stain was streak plated onto selective agar to allow for isolation of single colonies, which were then removed, and grown in 50 ml tryptic soy broth (TSB) for 24 hours at 37° C. or 30° C. in MRS broth for 48 h in the case of Lactobacillus. The cells were harvested by centrifugation (Sorvall™ ST 8) (5000 g for 10 min) and pellet resuspended in saline to a final cell density of 8 log cfu/ml, vortexed (IKA™ Vortex 3 Shaker) for one minute and stored at 4° C. for 48 hours, to allow for stress adaptation. The supernatant was discarded.

Non-waxed apples and whole head of iceberg lettuce were provided and stored at 4° C. until required. It was important that the produce used was intact without obvious signs of mechanical damage such as bruising and abrasions. Therefore, apples with any visible signs of damage (bruises, cuts, missing stems) or any spoilage were not used. The lettuce heads were prepared for treatment by removing the outermost layers of leaves which have had mechanical damage during processing.

Apples were spot inoculated on the skin, around the top of the fruit, with 100 μl of the test bacterium at a concentration of 8-log ₁₀CFU/ml, then allowed to dry in a biosafety cabinet for 20 min to 4 h then transferred to 4° C. for a maximum of 24 h. To internalize the bacteria, 1 ml of the suspension was added to the stem crevice and put under a vacuum for 1 minute, removed from the vacuum and left for 1 minute, before being vacuumed once more for another minute. Bacteria were recovered from apples by placing the whole fruit in a plastic pouch along with 100 ml of saline. The fruit was manually massaged for 60 s and a dilution series prepared in saline. L. monocytogenes was enumerated on Modified Oxford Formula agar (MOx) with Lactobacillus being plated onto MRS agar. In both cases plates were incubated at 30° C. for 48-72 h then typical colonies counted. The lower detection limit in both cases was 3 log cfu/apple.

Inactivation of Listeria monocytogenes and Lactobacillus Inoculated Onto Apples and Treated With Ozone

Apples were inoculated with either L. monocytogenes or Lactobacillus as described above. The inoculated apples (5 inoculated with Listeria and 5 with Lactobacillus) were then placed in PVC biobubble and exposed to ozone at a rate of 6 g/h. Upon completion of the treatment the survivors of ozone treatment were recovered as described above and log count reduction relative to controls determined.

### Laboratory Scale Forced Air Ozone Reactor

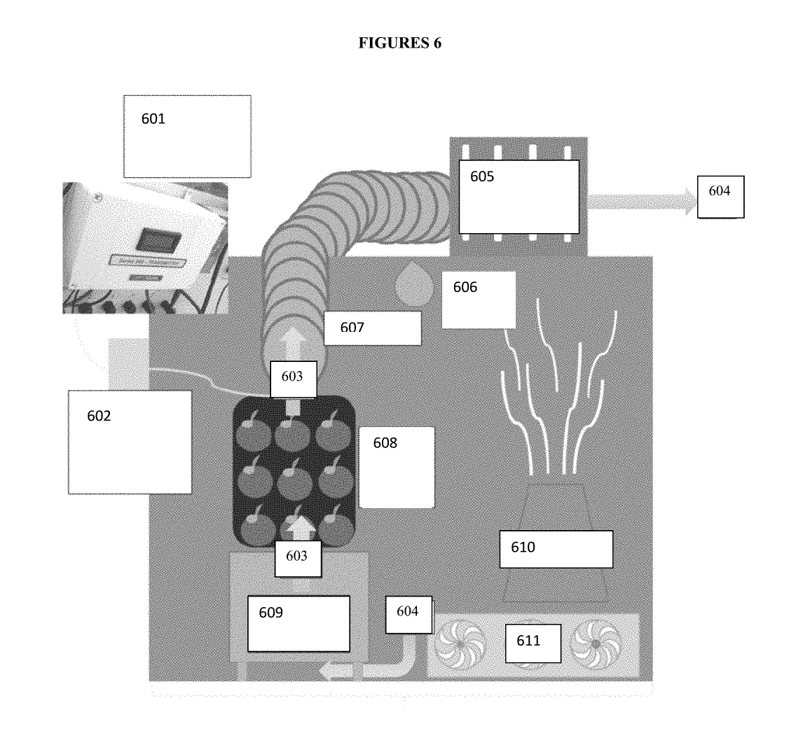

The reactor consisted of an ozone generator (Netech™, ozone output 6 g/h, flow rate 10 l/minute, power—120 W, 50/60 Hz) that was positioned at the base or top of a container (3.5′×3.5′×3.5′-½″ plywood box lined with 0.157″ corrugated plastic), sealed and/or closed about its perimeter and open at its top and bottom, into which apples were placed (30 cm depth) in a perforated box (FIG. 6). In FIG. 6, the following legends are used: 601: Series 940 transmitter Aeroqual; 602: Probe—temperature, ozone concentration and humidity; 603: ozone gas; 604: oxygen gas; 605: 4 UV lamps (254 nm) and exhaust fan; 606: heat lamp; 607: exhaust; 608: apples (30 cm depth); 609: ozone generator; 610: humidifier; and 611: fan.

The ozone was pulled up or down through the apple pile via a fan at a velocity of 9.5 m/s (measured with a CFM/CMM Thermo-Anemometer—Extech™—Model # AN100-20 point average for air flow and 3% velocity accuracy). The reactor was an enclosed system with the humidity being poised at 65-85% relative humidity via a humidifier (Honeywell # 3043-5974-0, 1-gallon capacity, 36 hour run time, low-high settings). Temperature, humidity and ozone concentration was measured using a Aeroqual series 940 monitoring unit (Auckland, NZ) calibrated by Aeroqual to a certified accuracy of <±0.008 ppm 0-0.1 ppm, <±10% 0.1-0.5 ppm.

The air was exhausted from the chamber via a fan and passed over 4 UV-C lamps (254 nm) to decompose residual ozone after treatment. The temperature of the apples was recorded using a thermometer probe (Fisher Scientific™ Traceable™—accuracy ±0.05° C.—range −50° to +150° C.) placed 0.5 or 1.0 cm into an apple fruit positioned in the middle of the pile. The treatment time was set for 20 min after which the apples were removed then sub-divided into those at the top, middle or bottom of the pile. The surviving L. monocytogenes was recovered as described above and enumerated on MOx with the initial loading being determined using non-treated fruit.

After the produce was inoculated and the pathogens allowed time to adhere, as described above, they were then placed in a chamber and exposed to ozone at a rate of 6 g/h. The parameters were controlled within the chamber as mentioned, with the ozone concentration starting from 30 ppm at five minutes of generation up to 80 ppm after 20 minutes. Humidity was stable from 85-90%, as kept constant by the humidifier, and temperature from 24.4° C. to 26.8° C. Upon completion of the treatment, residual ozone was broken down by 4 UV lights (254 nm wavelength—peak ozone destruction) and removed from the chamber with fans. The survivors of ozone treatment were recovered (described below) and log count reduction relative to controls determined.

### Effect of Air Flow on Efficacy of Ozone Decontamination of Apples

To determine if the point of introduction of ozone impacted the process, efficacy trials were undertaken where the gas was introduced into the chamber at different locations. In one arrangement, the ozone generator was placed on the bottom of a perforated container with 30 cm depth of apples. Three inoculated apples were placed at the base, middle or top. The ozone was drawn through the apple bed via a blower then passed the exhaust air over UV lamps to degrade residual ozone. In another arrangement the ozone was placed above the apples (outside of the chamber) with the air flow being forced downwards. Humidity, temperature and ozone concentrations were kept at constant rates as described above.

Efficacy of Ozone to Inactivate L. monocytogenes on Apples in Multiple Layers

Apples were inoculated with L. monocytogenes and left to attach for 2 hours at room temperature (21° C.). Inoculated apples (n=3) were then placed in the center row (B) in the tray (bottom) and the layer completed with non-inoculated apples. A further layer of inoculated apples (n=3) were place on the bed of apples (middle) and again layer completed with non-inoculated fruit. Finally, 3 inoculated apples were placed on the dual layer and surrounded by non-inoculated apples (top). Therefore, the tray had 3 layers of apples in total. The apples were placed in the chamber then treated with ozone for 20 minutes under high relative humidity for 20 minutes.

### Ozone Treatment of Conditioned Apples

Trials were performed to determine efficacy of ozone on apples with and without condensate. The apples were inoculated with Listeria with one set being placed at 4° C. for 12 hours with the other being held at 20° C. The apples were removed from 4° C. then placed directly in the treatment chamber and ozone (6 ppm) applied for 20 minutes.

### Commercial Scale Forced Air Ozone Generator Reactor

Apples were inoculated with 7 log cfu Lactobacillus suspension and transported in a cooler to facility. The reactor consisted of a generator (Medallion Indoor Environmental, model 03-20-24 UV Ultra High Output—twenty 24″ AT987 ozone lamps, 224/240 volt AC 50/60 HZ 8 amp, max ozone output 161.2 g/h, maximum air capacity 1200 CFM) placed at the top of the 4.0′×3.5′×10.0′—stainless steel unit that introduced ozone at a rate of 60 g/h (37 ppm) into the stainless steel chamber.

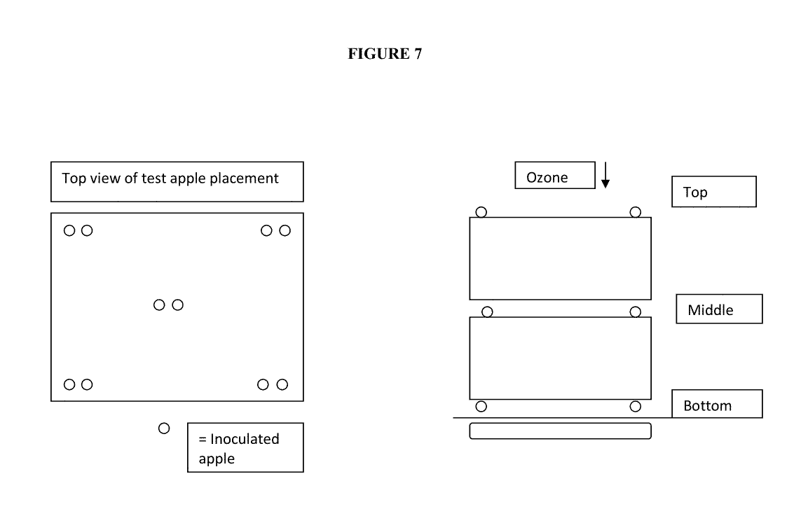

The apples within bins were held in a cooler prior to use and transferred to the treatment chamber directly to ensure condensate formed on the fruit surface. Two bins (3.9′×3.3′×2.5′) of apples were used for each trial that were stacked on top of each other and wrapped with plastic film to contain the ozone within the apple stack. A seal was formed on the top of the bin by the lid of the ozone delivery nozzle with the air velocity being controlled by an exhaust fan positioned at the base of the reactor (FIG. 7). The ozone concentration was measured at close proximity to the ozone exhaust port using an ozone monitor (2B Tech™, model 106-L, range 0-100 ppm ozone, accuracy 1.5 ppb). The concentration of ozone within the chamber ranged from 50 ppm-100 ppm. The treatment time and fan speed was set electronically along with an evacuation step upon completion of the process, a fan drawing the ozone through four 25 W lamps (measured at 254 nm at 100 hours and 80° F., 24″ long and 15 mm diameter—Standard UV lamps (serial # 05-1348)).

The inoculated apples were arranged at the top, middle or bottom of the chamber. Upon completion of the process the apples were removed and surviving Lactobacillus enumerated.

### Bacteria Recovery and Enumeration

### Lettuce

After treatment, lettuce heads were chopped, suspended in 500 ml of saline and stomached for 1 minute, a dilution series was prepared in saline. To enumerate STEC, the samples were then spread plated onto MacConkey Sorbitol agar (CT-SMAC) and chromogenic culture media (CHROMagar) incubated at 37° C. for 24 hours. L. monocytogenes was plated onto Modified Oxford Agar (MOX) incubated at 35° C. for 24-48 hours.

### Apples

After having challenges recovering pathogens from apples in the same manner as lettuce, baseline studies were performed in order to determine the suitable method for recovering Listeria from the surface of apples. The apples were spot inoculated with 100 μl of Listeria [8-log CFU] then allowed to attach for 4 hours. The Listeria was then recovered by one of three methods to evaluate the efficiency of each method. The methods were as follows: method (1) whole apples were placed in sterile plastic pouches and suspended in 100 ml of saline and manually rubbed for 1 minute. For method (2) a peeler was used to remove the apple peel which was then placed in 50 ml of saline and vigorously shaken for 1 minute. Lastly, method (3) was the same as described for (2) except the peel was homogenized using a lab top blender. Regardless, of the method of recovery, a dilution series was prepared in saline then spread plated onto Modified Oxford Agar (MOX) incubated at 35° C. for 24-48 hours. Presumptive positive colonies were counts being reported a log CFU.

### Effect of Listeria Incubation Temperature on Attachment

To determine if the incubation temperature of Listeria is important for its attachment to apples, the bacteria was cultivated at both 25° C. (were Listeria express flagella) and at 37° C. (i.e. no flagella expressed). The bacteria were allowed time to adhere to the apple before being removed (method 1) as described above.

### Statistical Analysis

Each experiment was repeated at least three times with triplicate samples being analyzed. The bacterial counts transformed into log₁₀ values with differences between means performed using ANOVA in combination with the Tukey test.

### Results

Suitability of Lactobacillus fructivorans as a surrogate for Listeria monocytogenes

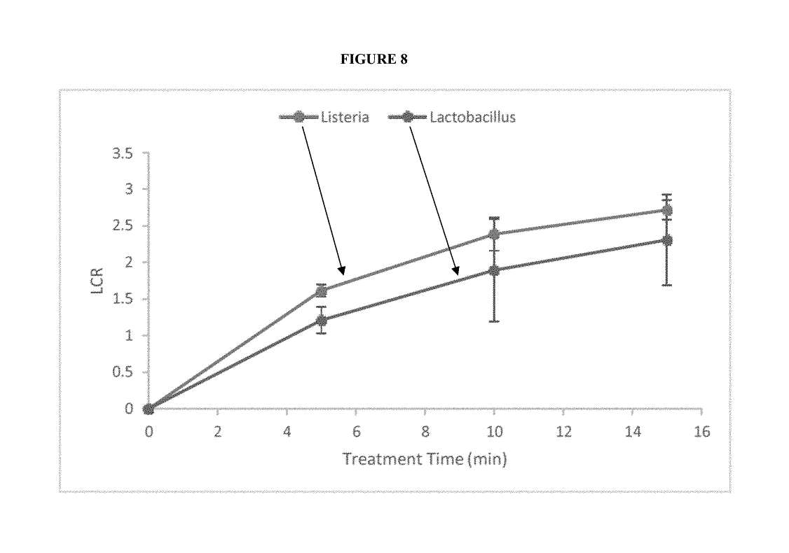

The relative resistance of Lactobacillus to ozone compared to L. monocytogenes, was assessed using inoculated apples placed inside a biobubble in which the antimicrobial gas was introduced. It was found that the extent of inactivation of Lactobacillus and L. monocytogenes by ozone treatment was dependent on the applied time (ozone concentration). In relative terms there was no significant difference (P>0.05) in the log reductions of L. monocytogenes compared to Lactobacillus receiving the same ozone exposure (FIG. 8). Therefore, the Lactobacillus strain is a suitable surrogate for L. monocytogenes that can be applied in commercial trials for accessing the efficacy of ozone treatment.

Effect of Air Flow Direction on the Efficacy of Ozone to Inactivate Listeria monocytogenes Inoculated into Apples

Inoculated apples were placed in the laboratory scale reactor then treated with ozone either by the gas being introduced at the top or bottom of container. The relative humidity was held between 65-85% relative humidity and treatment time set for 20 mins (Table 1).

It was found that the log count reductions of Listeria was independent on the position of the apple within the pile and also if the ozone was introduced at the top or base of the bed (Table 1). The results indicate that ozone can successfully infuse through the apple bed thereby enabling homogenous contact with the fruit regardless of the air flow direction.

TABLE 1
Log count reductions of Listeria inoculated onto apples
then treated in the Top or Bottom reactor as shown in FIG. 6.
Treatment was performed for 20 min with apple fruit initially
stored at 4° C. prior to loading into the reactor. Here the
mean of the samples is reported followed by the standard
error (the standard deviation divided by the square root of
the sample size n, where n is ≥ to 3).

| | Upward | Downward |
|---|---|---|
| Location of inoculated | Ozone Flow | Ozone Flow |
| apple within the batch | Listeria Log Count Reduction | |
| Bottom | 2.12 ± 0.94^{a} | 2.54 ± 0.37^{a} |
| Middle | 2.62 ± 0.80^{a} | 2.63 ± 0.96^{a} |
| Top | 2.55 ± 0.25^{a} | 3.07 ± 0.45^{a} |

Means followed by the same letter are not significantly different.

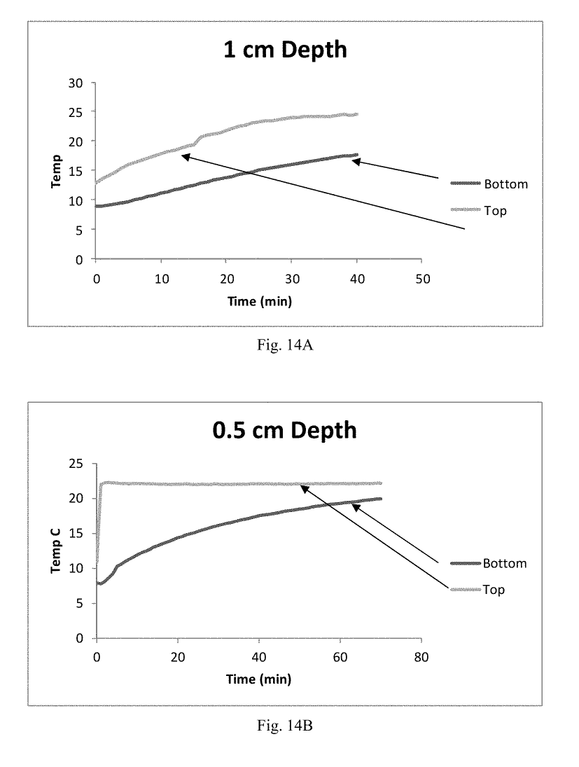

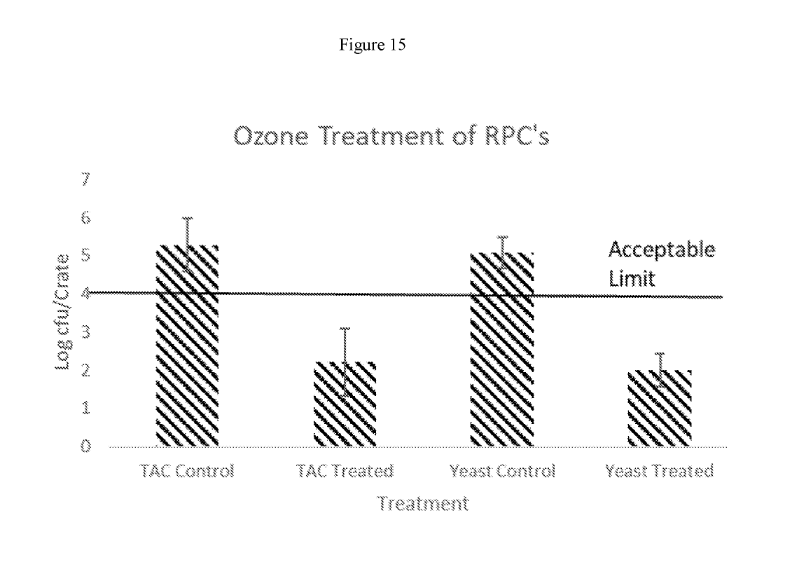

Although contact of ozone with apples was independent of the location of fruit within the pile there were differences with respect to the temperature profiles of fruit within the bed. Specifically, apples that received the incoming ozone stream warmed up quicker than those at the base (FIGS. 14A and 14B). The temperature at 0.5 cm depth of apples increased quicker compared to 1 cm into the fruit. The significance of the result is that the surface of the apple would retain moisture (condensation), provided a temperature differential exists. The rapid temperature increase of apples at the top of the pile would cause a higher rate of moisture removal compared to those at the base that in turn could reduce the efficacy of ozone. However, this was not the case according to comparable log count reductions of Listeria that was obtained irrespective of the position of the apple within the bed. It is possible that the surface apples would be exposed to a higher concentration of ozone that would compensate for the decrease in surface moisture.

### Commercial Scale Forced Air Ozone Reactor

A commercial scale reactor was constructed as described earlier, based on the findings of the laboratory trials. From an engineering prospective it was easier to introduce the ozone at the top of the unit then draw it down through the apple pile and exhaust at the bottom of the chamber. In validation trials, the inoculated apples were place in different positions in the apple pile to determine if the ozone treatment was being applied uniformly onto the apples. The ozone concentration was determined by ozone monitors as described above and the airflow monitored with an anemometer (as described above) which measures a combination of air velocity and volumetric measurements over a set period of time in cfm, with 1 cubic foot equaling approximately 28 liters.

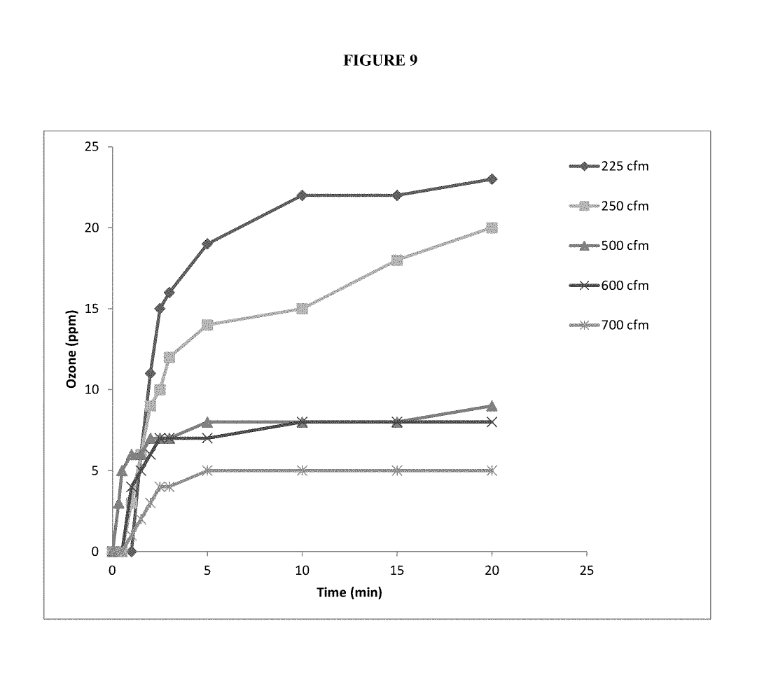

FIG. 9 shows effect of exhaust air velocity (cubic feet per minute) on the ozone concentration within the forced air ozone reactor. Two bins of apples were placed in the reactor and speed of the exhaust air fan set to give different air velocities. The ozone was introduced at the top of the reactor and measured after passing through the apple bed. The treatment was performed for a 20 minute period with the ozone concentration being logged every 30 seconds.

The air velocity at different parts of the reactor were measured using an air flow meter placed at different positions. By using a set air flow setting at 500 cfm the intake at the ozone inlet was 0.08 m³/s that decreased to 6.6×10⁻³ m³/s at the bottom of the apple pile and 0.27 m³/s at the air exit. The change in air velocity at different parts of the reactor is reflective of the diameter/area of the inlet, bed and outlet.

The ozone concentration measured near the air exhaust port was dependent on the air velocity (FIG. 9). At low exhaust air velocity the ozone concentration stabilized 10 mins into the run and attained the highest gas concentration. As the air velocity increased the level of ozone within the chamber decreased as did the time to achieve stable concentrations of the antimicrobial gas. At the highest exhaust air velocity (700 cfm) the ozone concentration recorded was 4 ppm that was significantly lower compared to when slower fan speed was applied.

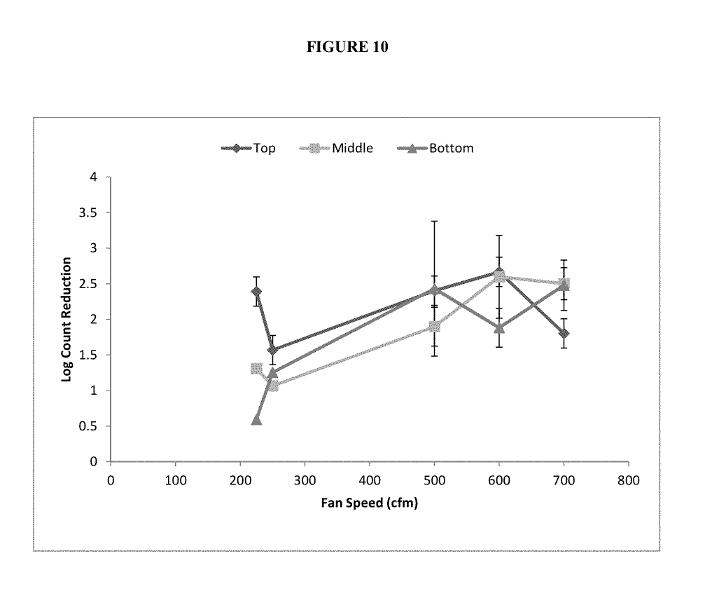

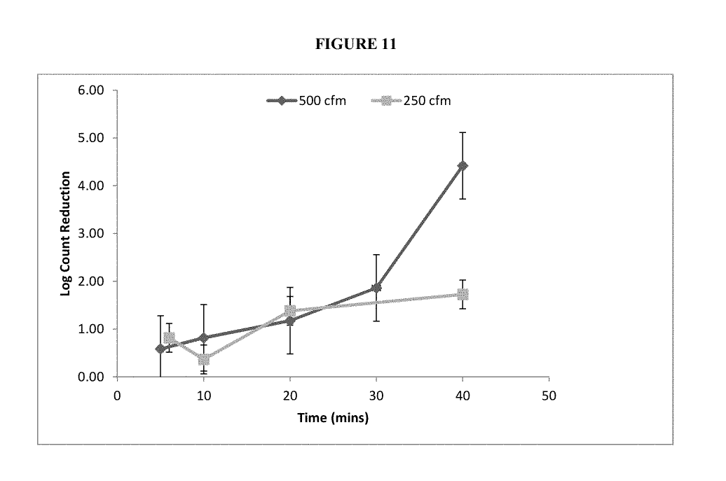

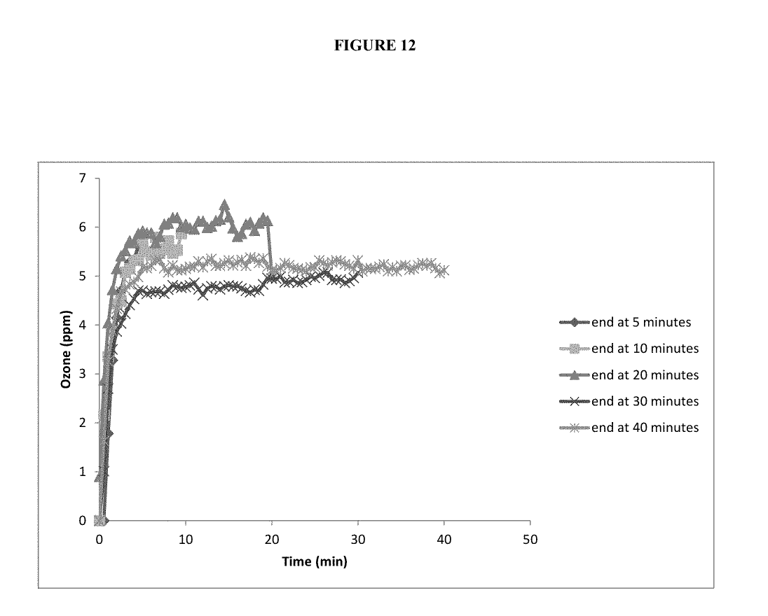

At low fan exhaust air velocity the log count reduction of Lactobacillus inoculated onto apples was dependent on the position of the apple within the pile. Specifically, a significantly higher log count reduction was obtained for those apples at the top of the pile compared those at the base. However, as the air velocity increased beyond 500 cfm there were no significant differences in terms of log count reduction of Lactobacillus at the top compared to the bottom of the apple pile. At the highest fan speed tested (700 cfm) the log reductions of Lactobacillus were significantly lower at the top of the apple pile compared to those positioned in the middle or at the bottom of the pile. Hence, the preferred exhaust air velocity is within the 500-600 cfm range. The effect of air exhaust velocity is likely due to a combination of ozone concentration and the dynamics of flow around the apple pile. At low exhaust fan speed the ozone would primarily accumulate at the top of the bed then slowly pulled through. As the fan speed increases the air being pulled through the generator dilutes the concentration of ozone but the flow through the bed is more homogenous. At the highest fan speed (700 cfm) the air being pulled through the ozone unit causes high dilution to the point that the concentration reduces in biocidal activity although can accumulate in the main body of the apple pile. Regardless of this fact, the preferred air exhaust speed lies between 500-600 cfm (FIG. 10).

FIG. 10 shows the Log count reduction of Lactobacillus on apples placed at the Top, Middle or Bottom within a forced air ozone reactor operating under different air exhaust velocities.

Apples were spot inoculated with Lactobacillus around the stem end the 5 fruit placed on top level of the apple pile, 5 in the middle and 5 under the bottom bin. Ozone was introduced at the top and drawn through the apple pile (2 bins) at different rates set by the exhaust fan. After 20 min treatment the apples were removed and Lactobacillus recovered.

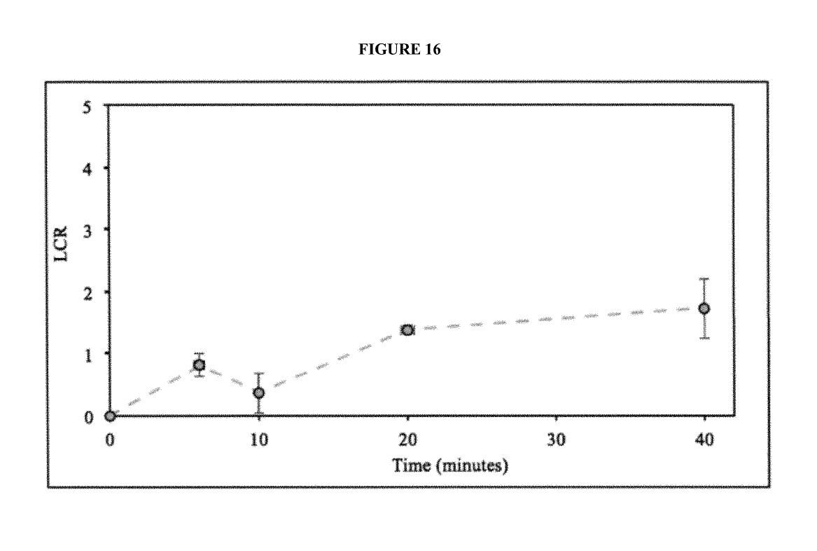

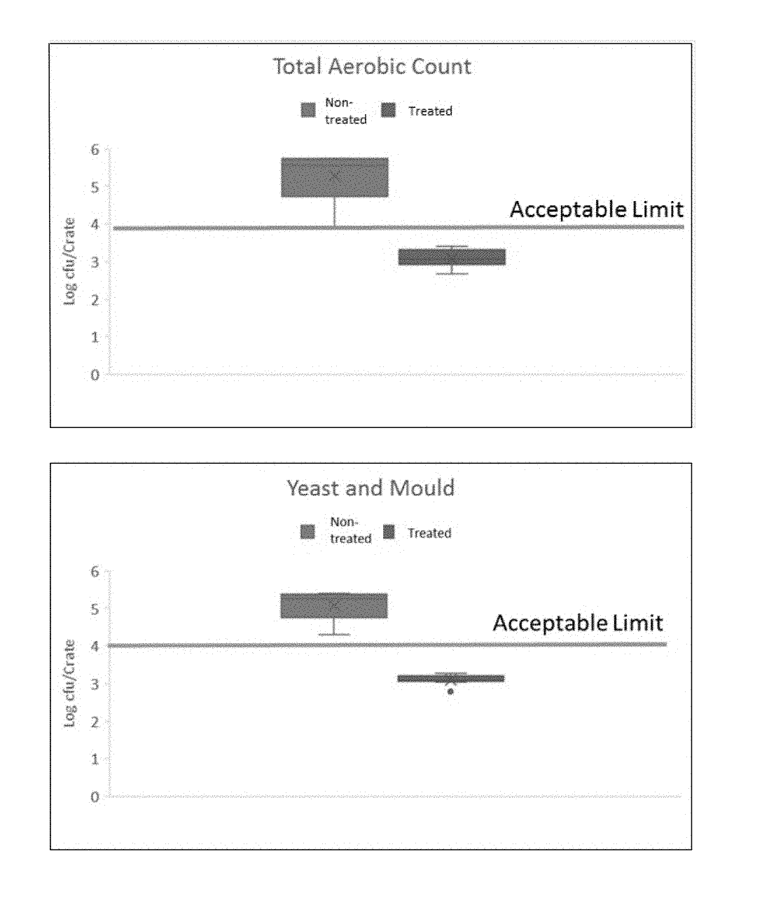

Trials were performed using an exhaust air velocity of 500 cfm to assess the effect of treatment time on the efficacy of the ozone mediated inactivation of Lactobacillus inoculated onto apples (FIG. 16). It was found that 6 or 10 min treatment times were not significantly different compared to controls, where air was pulled through the apple pile without ozone (0.19±0.29 log CFU reduction). However, treatment times >20 minutes supported a log reduction that was significantly greater compared to lower times. Increasing the treatment time to 40 minutes did not significantly increase the recorded log count reduction.

Trials were performed using an exhaust air velocity of 250 and 500 cfm to assess the effect of treatment time on the efficacy of the ozone mediated inactivation of Lactobacillus inoculated onto apples. It was found that for trials performed at 250 cfm, 6 or 10 min treatment times were not significantly different compared to controls where air was pulled through the apple pile without ozone (0.19±0.29 log cfu reduction). However treatment times >20 min supported a log reduction that was significantly greater compared to lower times. Increasing the treatment time to 40 mins did not significantly increase the recorded log count reduction.

In a similar manner, trials performed using the higher fan exhaust velocity (500 cfm) did not result in significant changes in Lactobacillus numbers at 5 or 10 mins. However, the log reductions of Lactobacillus were then found to increase with time. The longest treatment applied (40 min) resulted in a 4.42 log cfu reduction in Lactobacillus numbers.

While not wishing to be bound by theory, the inventors believe that the effectiveness of the treatment is not only related to predetermined exhaust air velocity, but also is connected to pressure differential through the bed of product/containers, as well as to turbulence at the surface.

### Discussion/Conclusion:

The ozone introduction rate is established such that a relatively steady concentration at about 14-20 ppm ozone within the chamber is achieved. Taking the data as a collective, the suitable processing conditions to decontaminate apples at such ozone concentration would be a 10-40 minute, preferably 15-25 minute, and most preferably about 20 minute treatment time; with a fan exhaust velocity of 500-600 cfm. The conditions would lead to a homogenous distribution of ozone within the bed whilst supporting an average 4.42 ±0.30 log cfu reduction of Listeria surrogate throughout the apple pile.

### EXPERIMENT 2

### Decontamination of Reusable Plastic Crates (RPC's) Using a Forced Air Ozone Reactor

### Materials and Methods

Pesudomonas fluorescens was cultivated in TSB for 24 h at 30° C. and cells harvested by centrifugation then resuspended in saline to an OD₆₀₀=0.2. Areas (2-3 cm²) were marked on the inside base and sides on unused RPC's onto which 0.1 ml of Pseudomonas suspension was deposited. The RPC's were kept at 23° C. and the inoculated areas sprayed with 10 ml volumes of TSB (per marked area) for a total of 5 days to support biofilm formation. In addition, Pseudomonas was inoculated 4 h before the ozone treatment to compare the inactivation of freshly deposited cells.

### Forced Air Ozone Treatment

Two trials were performed with the first placing one open crate and one collapsed on bins of apples. The bins were placed in the ozone reactor and treated for 40 mins. In the second trial the RPC's were placed at different locations within a stack that was arranged as would be delivered to fruit and vegetable packers. The stack of collapsed RPC's were placed in the ozone reactor and treated for 30 mins.

In addition to the inoculated RPC's, non-inoculated crates were samples by taking sponge samples from the interior. Ten randomly selected RPC's were sampled before ozone treatment and a different set of 10 after treatment.

### Microbiological Analysis

Sponges were used to recover Pseudomonas from the inoculated areas on the base and sidewalls of the crate. A separate set of crates that had not received ozone treatment were used to determine initial levels. The sponges were suspended in 30 ml of saline and homogenized by stomaching for 60 s. The homogenate was used to prepare a dilution series that as subsequently plated onto Pseudomonas agar that was incubated for 48 h at 30° C. In the event that no colonies were recovered the homogenate was added to an equal volume of TBS and incubated for 24 h at 30° C. The enriched culture was streaked onto Psudomonas agar and incubated at 30° C. for 24 h after which was inspected for typical colonies.

The sponge samples from non-inoculated RPC's were suspended in 30 ml of saline and homogenized by stomaching. A dilution series was prepared and plated out on TSA that was incubated at 34° C. to determine the total aerobic count and onto Potato Dextrose Agar incubated at 25° C. for 5 days to determine yeast & mold counts.

### Results

### Inoculated RPC's

| | | Log cfu/Crate | |
|---|---|---|---|
| | | (#Positive by | Log Count |
| | | Enrichment/Total Tested) | Reduction |
| Biofilm | | | |
| Initial Loading | Base | 3.56 ± 0.18 | |
| | Side | 3.55 ± 0.19 | |
| Open RPC | Base | (0/2) | 3.56 |
| | Side | (0/4) | 3.55 |
| Closed RPC | Base | (2/2) | 2.56 |
| | Side | (0/4) | 3.55 |
| Stack | | | |
| Top | Base | (0/2) | 3.56 |
| | Side | (2/4) | 3.05 |
| | Base | (0/4) | 3.55 |
| | Side | (0/2) | 3.56 |
| Middle | Base | (2/2) | 2.56 |
| | Side | (2/4) | 3.05 |
| | Base | (0/2) | 3.56 |
| | Side | (0/4) | 3.55 |
| Bottom | Base | (0/2) | 3.56 |
| | Side | (2/4) | 3.05 |
| | Base | (0/4) | 3.55 |
| | Side | (0/2) | 3.56 |
| Fresh Inoculated | | | |
| Initial Loading | Base | 6.42 ± 0.29 | |
| | Side | 5.46 ± 1.08 | |
| Open RPC | Base | (0/2) | 6.42 |
| | Side | (0/4) | 5.46 |
| Closed RPC | Base | (2/2) | 5.92 |
| | Side | (0/4) | 5.46 |
| Stack | | | |
| Top | Base | (0/2) | 6.42 |
| | Side | (0/4) | 5.46 |
| | Base | (0/2) | 6.42 |
| | Side | (0/4) | 5.46 |
| Middle | Base | (1/2) | 5.92 |
| | Side | (4/4) | 4.46 |
| | Base | (2/2) | 5.42 |
| | Side | (2/4) | 4.96 |
| Bottom | Base | (2/2) | 5.42 |
| | Side | (2/4) | 4.96 |
| | Base | (2/2) | 5.42 |
| | Side | (4/4) | 4.46 |

### Non-Inoculated RPC's

See FIG. 15

### Conclusions

The forced air ozone reactor treatment could reduce Pseudomonas in biofilms and on the surface of RPC's to levels low enough to only be detectable by enrichment. In general, the decontamination efficacy of ozone treatment was independent on the position of the RPC's within the stack although a higher frequency of positive samples were detected in those crates positioned in the middle and bottom of the stack. Ozone treatment reduced the levels of endogenous microbial levels below the level of acceptability with regards to total aerobic counts and yeast and molds.

1. A method for inactivating bacteria and/or reducing microbial count on a food product or a container therefor, which is susceptible to surface and sub-surface microbial presence, said method comprising
a) providing a plurality of said food product or container in a sealable chamber which is operably connected to i) an ozone generator for generating ozone gas, and ii) an evacuation fan for forcing movement of ozone gas vertically through the sealable chamber;
b) creating condensation on surface of the food product or container by adjusting humidity in the sealable chamber to reach a predetermined humidity;
c) operating the ozone generator and the evacuation fan to generate a predetermined exhaust air velocity to pass ozone gas generated by the ozone generator through the sealable chamber for a predetermined period of dwell time; and
d) expelling ozone gas from the sealable chamber.

2. The method of claim 1, wherein the bacteria is Listeria, Salmonella or E. Coli.

3. (canceled)

4. The method of claim 1, wherein the food product is apple, melon, lettuce, mushroom, zucchini, cucumber, beehive, seed, spice, tea, grain, dried fruits or nuts.

5. (canceled)

6. (canceled)

7. The method of claim 1, wherein the predetermined humidity is about 70-100%.

8. The method of claim 1, wherein the dwell time is greater than 10 minutes.

9. (canceled)

10. The method of claim 1, wherein the predetermined exhaust air velocity is about 10-1500 cfm.

11. (canceled)

12. The method of claim 1, wherein ozone concentration in the sealable chamber is maintained at about 4-20 ppm in step c) for a period of time sufficient to kill from 99-99.999% of the bacteria.

13. (canceled)

14. The method of claim 1, wherein the sealable chamber has capacity to hold 1-3000 lbs.

15. (canceled)

16. The method of claim 1, wherein the sealable chamber has capacity to hold at least 1 lb of food product.

17. The method of claim 1, said method excluding a step of contacting an ozone-containing liquid with the food product or container.

18. The method of claim 1, wherein ozone is introduced into the sealable chamber at rates of about 1-60 g/h.

19. The method of claim 1, wherein:
(i) the predetermined humidity is about 70-100%,
(ii) the dwell time is greater than 10 minutes,
(iii) the predetermined exhaust air velocity is about 10-1500 cfm, and
(iv) ozone concentration in the sealable chamber is maintained at about 4-20 ppm in step c).

20. An apparatus for inactivating bacteria and/or reducing microbial count on a food product or a container therefor, which is susceptible to surface and sub-surface microbial presence, said apparatus comprising a sealable chamber which is operably connected to i) an ozone generator for generating ozone gas, and ii) an evacuation fan for forcing movement of ozone gas vertically through the sealable chamber.

21. The apparatus of claim 20, which further comprises an ozone sensor.

22. The apparatus of claim 20, which further comprises a dryer assembly, said dryer assembly comprising a hood and a dryer exhaust fan.

23. The apparatus of claim 20, wherein the sealable chamber has capacity to hold 1-3000 lbs.

24. The apparatus of claim 23, wherein the sealable chamber has capacity to hold about 1600-3000 lbs of food product.

25. The apparatus of claim 20, wherein the sealable chamber has capacity to hold at least 1 lb of food product.

26. A method for reducing level of yeast, mold and/or mildew in a container, said method comprising
a) providing a one or more of said container in a sealable chamber which is operably connected to i) an ozone generator for generating ozone gas, and ii) an evacuation fan for forcing movement of ozone gas vertically through the sealable chamber;
b) creating condensation on surface of the one or more containers by adjusting humidity in the sealable chamber to reach a predetermined humidity;
c) operating the ozone generator and the evacuation fan to generate a predetermined exhaust air velocity to pass ozone gas generated by the ozone generator through the sealable chamber for a predetermined period of dwell time; and
d) expelling ozone gas from the sealable chamber.

27. The method of claim 26, wherein the container is a Reusable Plastic Containers (RPC).
